(19) United States
(12) Patent Application Publication
(10) Pub. No.: US 2009/0252812 A1
(43) Pub. Date: Oct. 8, 2009

(54) Use of Fermented Milk Product for Skin Treatment

(76) Inventors: Severine Lieurey, Dublin (IE); Stephen Watkins, Kent (GB)

Correspondence Address: NORRIS, MCLAUGHLIN & MARCUS, 875 THIRD AVE, 18TH FLOOR, NEW YORK, NY, 10022, US

(21) Appl. No.: 12/293,897

(22) PCT Filed: Mar. 21, 2007
(86) PCT No.: PCT/GB07/01003

§ 371 Date: Sep. 22, 2008

(30) Foreign Application Priority Data
Mar. 24, 2006 (GB) 0605949.7

(51) Int. Cl. A61K 8/98; A61Q 19/00; A61Q 19/08

(52) U.S. Cl. 424/535

(57) ABSTRACT

The use of a fermented milk product comprising non-hydrolysed whey proteins which is substantially free of casein proteins for the purpose of improving skin firmness, by structuring collagen without promoting collagen synthesis, when topically applied to skin is provided.

### TECHNICAL FIELD

The present invention relates to the use of a fermented milk product comprising non-hydrolysed whey proteins for treatment of skin, especially human skin.

### BACKGROUND AND PRIOR ART

Milk and dairy products have been used in cosmetic skin care applications for many hundreds of years as a way to add moisture and fats to the skin in order to condition and moisturise. Fermented milk (e.g. yoghurt) has also been used historically for skincare applications. Such yoghurts are typically high in casein content.

It is well documented that exposure to sunlight damages the skin structure. In response to this damage the skin repairs itself through the rapid production of collagen and other associated dermal components such as polysaccharides. Unfortunately, this rapid formation can result in an unstructured ‘mess’ of fibres, a condition termed elastosis. It is common for ingredients that show the ability to structure collagen to also trigger collagen synthesis, a result that is not always desirable.

WO 2004/098632 (Snow Brands) discloses cosmetic compositions containing inhibitors which promote skin collagen formation, containing milk basic protein fractions or their hydrolysates.

U.S. Pat. No. 6,203,805 (Color Access) discloses use of whey protein to enhance production of collagen in skin.

EP 0046326 A discloses use of non-hydrolysed whey products in cosmetics.

### SUMMARY OF THE INVENTION

The present invention relates to the use of a fermented milk product comprising non-hydrolysed whey proteins which is substantially free of casein proteins for the purpose of improving skin firmness when topically applied to skin.

The particular fermented milk products used according to the invention have been found to be effective at structuring collagen without promoting collagen synthesis. This surprising and unusual property has the result that skin firmness improves when the product is topically applied to the skin.

Substantially free of casein proteins means that the product comprises less than 5 wt %, preferably less than 3 wt %, more preferably less than 2 wt %, or even less than 1 wt % of casein proteins.

Thus, in a further aspect, the invention relates to the use of a fermented milk product comprising non-hydrolysed whey proteins which is substantially free of casein proteins for the purpose of structuring collagen without promoting collagen synthesis when topically applied to skin.

The novel property of the fermented milk products makes them particularly suitable for use on damaged skin when the skin will naturally generate collagen as part of the repair process.

Thus, in a further aspect, the invention relates to the use of a fermented milk product comprising non-hydrolysed whey proteins which is substantially free of casein proteins for application on damaged skin for the purpose of collagen structuring.

Preferably, the milk product contains low or no milk fats, typically achieved by fermenting skimmed milk. Low or no milk fats generally means that the milk product contains no more that 10 wt % milk fats, preferably no more than 5 wt % milk fats, more preferably no more than 2 wt % milk fats, or even containing zero milk fats.

The fermented milk products are typically yoghurt-based. The invention may use yoghurt, preferably made from semi-skimmed milk or skimmed milk. However, any yoghurt must be substantially free of casein proteins. The invention may alternatively use a range of different yoghurt-derived proteinaceous materials. In particular, the invention conveniently uses modified yoghurt, modified by treatment to remove substantially all of the casein proteins. Such modification has the benefits of producing material that is less allergenic (as allergenic effects are generally due to casein proteins), also removing some of the fat content, and producing material that is less prone to smelling (as rancid “off” smells associated with milk and milk-based products are generally due to casein proteins and/or fat). Yoghurt may also be further modified by addition of whey proteins to compensate for removal of casein proteins. Such modifications are suitably performed before the fermentation stage in the conventional process for producing yoghurt.

The fermented milk product is desirably used in dried condition for inclusion in a composition (e.g. in the form of a powder or granules) rather than in liquid condition. Use of a dried fermented milk product compared to one in liquid condition as an ingredient in a topically applied product has certain practical benefits, in particular in terms of a longer shelf life of the dried ingredient, lower content of micro-organisms and avoidance of risk of microbial growth. Material in dry condition can also be incorporated into powdered or anhydrous products.

The fermented milk product is conveniently dried, preferably by a spray drying technique. Suitable drying techniques are well known to those skilled in the art.

It is particularly preferred to use a spray dried modified yoghurt product known by the Trade Mark Yogurtene and available from Quest International as a food supplement. Yogurtene consists of selected milk fractions: whey, whey concentrates (components of the serum phase of milk) and non-fat dry milk (2%), that are fermented with classic yoghurt bacteria (Streptococcus thermophilus and Lactobacillus bulgaricus). Whey is the preferred protein source, because casein proteins are associated with allergenic reactions. Once fermentation is complete, the liquid yoghurt is spray dried to produce a free-flowing, slightly hygroscopic, low odour white to off-white powder.

The fermented milk product is desirably substantially free of live bacteria, i.e. it contains no more than 1000 cfu/g, preferably no more than 500 cfu/g, more preferably no more than 200 cfu/g, or even zero live bacteria. Bacteria can be conveniently destroyed by heat treating the milk product in a manner known to a person skilled in the art.

The fermented milk product used in the present invention is typically applied to the skin, in particular human skin, in a topically applied product. Examples of suitable products are body wash compositions, soap-based products, skin creams and lotions, sunscreens and after-sun products, and in particular, anti-ageing compositions which may also include components for treating cellulite, sagging, wrinkling, age spots and the like. Other suitable products are cosmetics such as lipsticks, foundations and the like.

The amount of fermented milk product in a topically applied product will vary depending upon the application but will generally fall within the range of from 0.005 to 20 wt %.

The invention will now be further described, by way of illustration, in the following examples:

### EXAMPLE 1

### Collagen Fibre Organisation

Collagen is a major constituent of the human skin and accounts for a high proportion of the skin's elasticity and physical properties. When collagen is incubated in vitro with normal human dermal fibroblasts (NHDF), the NHDF will spontaneously bind to the soluble collagen and organise it into a structured lattice. The resulting structure visibly shrinks as this process occurs, and the level of shrinkage is directly proportional to the amount of structuring that occurs.

The rate of collagen organisation can be influenced by the addition of different ingredients into the system. Therefore, it can be shown that some materials will enhance the rate of the lattice formation and so could have a ‘firming’ effect if applied topically to the skin.

A collagen solution was prepared in vitro (1.3 mg/ml) and incubated with NHDF (100,000 NHDF/ml) over a 168-hour period (control). The amount of ‘lattice shrinkage’ was monitored at 24 hours, 72 hours, 144 hours and 168 hours. Over this period, the same concentration of collagen/NHDF was cultured with Yogurtene™ at 0.08 mg/ml, 0.4% mg/ml and 2 mg/ml. A positive control, Transforming Growth Factor (beta-TGF), was used to compare to the lattice enhancing effects of Yogurtene™. Each test was conducted in triplicate and the mean shrinkage calculated.

Table 1 (below) shows the surface area of the collagen solution as measured over the experiment.

TABLE 1

| | | | 24 hr incubation | | 72 hr incubation | | 144 hr incubation | | 168 hr incubation | |
|---|---|---|---|---|---|---|---|---|---|---|
| | | | Surface | | Surface | | Surface | | Surface | |
| | | | Area | | Area | | Area | | Area | |
| Treatment | | | (mm²) | mean | (mm²) | mean | (mm²) | mean | (mm²) | mean |
| Control | — | | 590.70 | 557.20 | 464.70 | 443.54 | 332.10 | 328.57 | 264.39 | 266.50 |
| | | | 549.52 | | 433.36 | | 309.79 | | 258.96 | |
| | | | 531.37 | | 432.56 | | 343.81 | | 276.14 | |
| beta-TGF | 10 | ng/ml | 589.89 | 586.75 | 456.84 | 436.64 | 192.89 | 193.24 | 107.19 | 110.27 |
| | | | 580.88 | | 416.06 | | 194.06 | | 121.69 | |
| | | | 589.49 | | 437.03 | | 192.76 | | 101.92 | |
| Yogurtene | 2 | mg/ml | 562.61 | 560.72 | 419.74 | 411.62 | 301.20 | 297.58 | 223.45 | 213.82 |
| | | | 545.66 | | 394.42 | | 292.20 | | 215.04 | |
| | | | 573.89 | | 420.69 | | 299.34 | | 202.96 | |
| | 0.4 | mg/ml | 559.88 | 549.48 | 450.62 | 442.17 | 336.42 | 332.79 | 243.58 | 243.46 |
| | | | 525.61 | | 422.87 | | 319.17 | | 234.32 | |
| | | | 562.96 | | 453.02 | | 342.78 | | 252.47 | |
| | 0.08 | mg/ml | 529.16 | 517.51 | 430.42 | 416.94 | 333.33 | 321.89 | 264.25 | 264.69 |
| | | | 511.60 | | 406.61 | | 309.85 | | 258.10 | |
| | | | 511.77 | | 413.79 | | 322.48 | | 271.73 | |

Thus Yogurtene™ shows noticeable collagen structuring.

### EXAMPLE 2

### Dermal Extracellular Matrix Synthesis

Normal human dermal fibroblasts (NHDF) were cultured in-vitro with radio labelled [³H] proline. Collagen is a proline-rich protein. The level of radio labelled proline found in proteins, following culturing of the NHDF, is a measure of the rate of collagen synthesis. Yogurtene™ can be added to the cell culture to measure the effect of Yogurtene™ on the rate of proline uptake (collagen synthesis).

The study investigated the increase in radio labelled proline found in: (i) soluble/secreted proteins, and (ii) intracellular and extra-cellular matrix (ECM) proteins.

Beta-Transforming Growth Factor (β-TGF) and ascorbic acid are known collagen synthesis promoters and were used as controls in both studies.

Tables 2 and 3 show the levels of proline found in the associated proteins.

TABLE 2
Proline incorporation into soluble proteins

| | | |
|---|---|---|
| Treatment | Concentration | Count/min |

| Control | — | | 8180 |
|---|---|---|---|
| β-TGF | 10 | ng/ml | 17303 |
| Ascorbic acid | 20 | micro g/ml | 17918 |
| Yogurtene ™ | 2 | mg/ml | 8759 |
| | 0.4 | mg/ml | 8589 |
| | 0.08 | mg/ml | 8363 |
| | 0.016 | mg/ml | 7992 |

TABLE 3
Proline incorporation into intracellular & ECM layer proteins

| | | |
|---|---|---|
| Treatment | Concentration | Count/min |

| Control | — | | 10325 |
|---|---|---|---|
| β-TGF | 10 | ng/ml | 18386 |
| Ascorbic acid | 20 | micro g/ml | 12764 |
| Yogurtene ™ | 2 | mg/ml | 11421 |
| | 0.4 | mg/ml | 11917 |
| | 0.08 | mg/ml | 10418 |
| | 0.016 | mg/ml | 11023 |

This data demonstrates that Yogurtene™ does not increase the synthesis of collagen.

1. A process for improving skin firmness, which process comprises the step of:
topically applying a fermented milk product comprising non-hydrolysed whey proteins which is substantially free of casein proteins to skin.

2. A process for structuring collagen without promoting collagen synthesis, which process comprises the step of:
topically applying a fermented milk product comprising non-hydrolysed whey proteins which is substantially free of casein proteins to skin.

3. A process collagen structuring of damaged skin, which process comprises the step of:
topically applying a fermented milk product comprising non-hydrolysed whey proteins which is substantially free of casein proteins on damaged skin.

4. The process according to claim 1, wherein the fermented milk product comprises no more than 10 wt % milk fats.

5. The process according to claim 1, wherein the fermented milk product comprises yoghurt or yoghurt-derived materials.

6. The process according to claim 1, wherein the fermented milk product is in dried condition.

7. The process according to claim 1, wherein the fermented milk product is substantially free of live bacteria.

8. The process according to claim 1, wherein the fermented milk product is a spray-dried modified yoghurt product substantially free of live bacteria and containing no more than 10 wt % milk fats.

9. The process according to claim 1, wherein the fermented milk product is applied in a topically applied product.

10. The process according to claim 2, wherein the fermented milk product comprises no more than 10 wt % milk fats.

11. The process according to claim 2, wherein the fermented milk product comprises yoghurt or yoghurt-derived materials.

12. The process according to claim 2, wherein the fermented milk product is in dried condition.

13. The process according to claim 2, wherein the fermented milk product is substantially free of live bacteria.

14. The process according to claim 2, wherein the fermented milk product is a spray-dried modified yoghurt product substantially free of live bacteria and containing no more than 10 wt % milk fats.

15. The process according to claim 3, wherein the fermented milk product comprises no more than 10 wt % milk fats.

16. The process according to claim 3, wherein the fermented milk product comprises yoghurt or yoghurt-derived materials.

17. The process according to claim 3, wherein the fermented milk product is in dried condition.

18. The process according to claim 3, wherein the fermented milk product is substantially free of live bacteria.

19. The process according to claim 3, wherein the fermented milk product is a spray-dried modified yoghurt product substantially free of live bacteria and containing no more than 10 wt % milk fats.
